(19) United States
(12) Patent Application Publication
(10) Pub. No.: US 2009/0292477 A1
(43) Pub. Date: Nov. 26, 2009

(54) Method and Apparatus for Screening and Assaying Environmental Sample

(76) Inventors: Takashi Matsuki, Kyoto-shi (JP); Kazuyuki Sawadaishi, Kyoto-shi (JP); Chiwa Kataoka, Kyoto-shi (JP); Yoko Takagi, Kyoto-shi (JP)

Correspondence Address: BIRCH STEWART KOLASCH & BIRCH, PO BOX 747, FALLS CHURCH, VA, 22040-0747, US

(21) Appl. No.: 11/922,123

(22) PCT Filed: Jun. 14, 2005
(86) PCT No.: PCT/JP2005/010832

§ 371 Date: Dec. 13, 2007

(51) Int. Cl. G01N 33/48; G06F 19/00

(52) U.S. Cl. 702/19; 73/61.41

(57) ABSTRACT

A screening measurement technology enabling a prompt determination about whether or not further confirmation is required by another measuring method, for an environmental sample, such as a sample containing dioxins, is provided. A sample solution in which a known amount of antibodies are mixed, is poured into a measurement cell (101) where an antigen derivative (107) that acquires antibodies of a subject substance comprising an antigen, is arranged in a flow channel (106) for the sample solution; whether or not a measurement result is within a pre-determined range including a reference value of the subject substance is determined based upon time-series signals of the measurement result; and the measurement result is transmitted.

### TECHNICAL FIELD

The present invention relates to a method and a device for screening measurement of environmental samples containing environmental pollutants, such as dioxins.

### BACKGROUND ART

Persistent organic pollutants (POPs) typified by dioxins adversely affect organisms over a long period of time. They are harmful, easily soluble into fat, and difficult to be decomposed even in the environment. Especially the dioxins are highly toxic, and even an infinitesimal quantity of dioxins are harmful in organisms.

Since many isomers exist in the dioxins and the toxicity varies depending upon the isomer, the toxicity of a sample is evaluated according to a toxicity equivalent quantity (TEQ). The toxicity equivalent quantity is obtained based upon the toxicity of 2,3,7,8-tetrachlorodibenzo-para-dioxin (2,3,7,8-TCDD), which has the highest toxicity among the dioxins. “The toxicity equivalent quantity” is the total of values obtained by multiplying the toxicity of each isomer when the toxicity of 2,3,7,8-TCDD is regarded as 1 by the abundance of each isomer.

For measuring dioxins, a method using a high-resolution gas chromatography mass spectrometery (HRGC/MS) has been adopted as the official method. In the official method, dioxins are extracted using a soxhlet extraction method and a cleanup operation is conducted for removal of measurement interference substances, during pre-treatment. A pre-treatment sample is injected into a capillary column of HRGC/MS. Isomers are separated by the capillary column. Each isomer eluted from the capillary column sequentially enters into a double-focusing mass spectrometery. Determination of data of dioxins according to a chromatograph of a mass spectrometer enables abundant obtainment per isomer. If a composition of dioxins can be determined, identification of a contaminant source becomes easier. Further, it is possible to figure out the toxicity equivalent quantity without being affected by the relative proportions of the isomers.

Other than the official method, a simple measuring method is also used for measuring dioxins. In the simple measuring method, the pre-treatment and the measuring technique are simplified, resulting in a reduction of a period of time required for obtaining a result, reducing the cost. As the method to simplify the measuring technique, a method to use a low-resolution gas chromatography mass spectrometery and a bioassay method are available.

For the bioassay method, a bioassay method by utilizing an Ah receptor and an immunoassay method by utilizing an antigen-antibody complex reaction are available. An ELISA (enzyme-linked immunosorbent assay) is one of the immunoassay methods obtained by utilizing an enzyme label. For example, a sample solution containing subject substances, such as dioxins, and a solution containing enzyme labels are mixed, and the mixed solution is placed in an antibody solid-phased plate. After the subject substances, which are antigens, and the enzyme labels bind with the antibodies, unreacted substances are removed by washing, and a chromogenic substance which will react with the enzyme label, is added. Measuring the absorbance of the sample under these conditions results in obtaining a concentration of the subject substances.

### DISCLOSURE OF THE INVENTION

As described above, the time required for obtaining the results is reduced by using the simple measuring method. Since approximately a few weeks are required for obtaining the result using the GC/MS method, a screening measurement is conducted using the simple measuring method.

However, even when the simple measuring method is used, it is difficult to determine whether or not further measurement is required using the GC/MS method or another method, making it difficult to make a determination.

The present invention has been accomplished by taking the problems in the related art into consideration, and has the objective of providing a method and a device for screening measurement of environmental samples enabling the prompt determination of selection.

In order to accomplish the objective, according to one aspect of the present invention, the screening measuring method is provided. The method comprises the steps of: measuring an environmental sample; determining whether or not the measurement results are within a pre-determined range including a reference value based upon time-series signals of the measurement result; and outputting the determined results.

According to another aspect of the present invention, a screening measuring method for an environmental sample is provided. The method comprises the steps of: pouring a sample solution in which a known amount of antibody of a subject substance being an antigen is mixed, into a measurement cell having a flow channel for the sample solution, wherein an antigen derivative for acquiring the antibody is arranged in the flow channel; measuring the antibody acquired by the antigen derivative; determining whether or not a measurement result is within a given pre-determined range including a reference value based upon a time-series signal of the measurement result; and outputting the measurement result.

In the screening measuring method, the step for determination can comprise the step of predicting whether or not the measurement result of the sample is within a pre-determined range based upon a time-series signal in the early of sample measurement, and the determination can be conducted based upon the predicted result.

Preferably, the screening measuring method further comprises the step of moving on to the measurement of another sample, in case of predicting that the measurement result of the sample is not within the pre-determined range.

In the step for output, in case of determining that the measurement result is within the pre-determined range in the step for determination, necessity to confirm whether or not to satisfy a reference value can be displayed.

According to another aspect of the present invention, a screening measuring method is provided. The screening measuring method comprises the steps of: pouring a sample solution in which a known amount of antibody of a subject substance being an antigen is mixed, into a measurement cell having a flow channel for the sample solution, wherein an antigen derivative for acquiring the antibody is arranged in the flow channel; measuring the antibody acquired by the antigen derivative; and displaying a pre-determined range including a reference value of the subject substance and a time-series signal of the measurement result to be comparable.

According to still another aspect of the present invention, a screening measuring device used for the screening measuring method described above can be provided. The screening measuring device comprises: a measurement cell that has a flow channel for a sample solution, wherein an antigen derivative for acquiring an antibody of a subject substance being an antigen is arranged in the flow channel; a unit configured to measure the antibody acquired by the antigen derivative by pouring the sample solution in which a known amount of the antibody is mixed, into the measurement cell; a unit configured to store data indicating a pre-determined range including a reference value for the subject substance; a unit configured to determine whether or not the measurement result is within the pre-determined range based upon a time-series signal of the measurement result; and a unit configured to output the measurement result.

In this screening measuring device, the unit for determination can predict whether or not the measurement result of the sample is within the pre-determined range based upon a time-series signal in the early of sample measurement and conduct the determination based upon the predicted result.

According to still another aspect of the present invention, a screening measuring device is provided. The screening measuring device comprises: a measurement cell having a flow channel for a sample solution, wherein an antigen derivative for acquiring an antibody of a subject substance being an antigen is arranged in the flow channel; a unit configured to measure the antibody acquired by the antigen derivative by pouring the sample solution in which known amount of the antibody is mixed, into the measurement cell; a unit configured to store data indicating a pre-determined range including a reference value for the subject substance; and a unit configured to display the pre-determined range including a reference value for the subject substance and a time-series signal of the measurement result to be comparable and the time-series signal of the measurement result.

By adopting these configurations, the present invention enables prompt selection determination of environmental samples.

### BRIEF DESCRIPTION OF DRAWINGS

FIG. 1 is a diagram for explaining an outline configuration of a screening measuring system in an embodiment of the present invention.

FIG. 2 is a diagram for explaining the measurement principle of the present invention.

FIG. 3 is a diagram showing one example of measurement data.

FIG. 4 is a diagram showing a correlation between the measuring method of the present invention and an official method regarding an exhaust gas sample.

FIG. 5 is a diagram showing a correlation between the measuring method of the present invention and the official method regarding a bottom ash sample.

FIG. 6 is a diagram showing a correlation between the measuring method of the present invention and the official method regarding a fly ash sample.

FIG. 7 is a diagram showing a correlation between the measuring method of the present invention and the official method regarding a soil sample.

FIG. 8 is a diagram showing one example of a monitor screen.

FIG. 9 is one example of a screen displaying a measurement result in the case of stopping the sample measurement halfway.

### DESCRIPTION OF THE NUMERALS

1 measuring part
2 data processor
101 measurement cell
102 pump
103 excitation light source
104 light power detector
105 controller
106 flow channel
107 solid-phased antigen derivative
203 HDD
205 CPU

### BEST MODE FOR CARRYING OUT THE INVENTION

An embodiment of the present invention is described hereafter, with reference to the drawings. In the embodiment, the present invention is embodied as a system for screening measuring of environmental samples containing dioxins, as subject substances.

The screening measuring system in the embodiment forms antigen-antibody complexes by reaction of a pre-treatment environmental sample containing dioxins with a fluorescently-labeled antibody solution; acquires unreacted antibodies in the reaction solution by a measurement cell filled up with the antigen-antibody solid-phased carrier; and measures the fluorescent intensity.

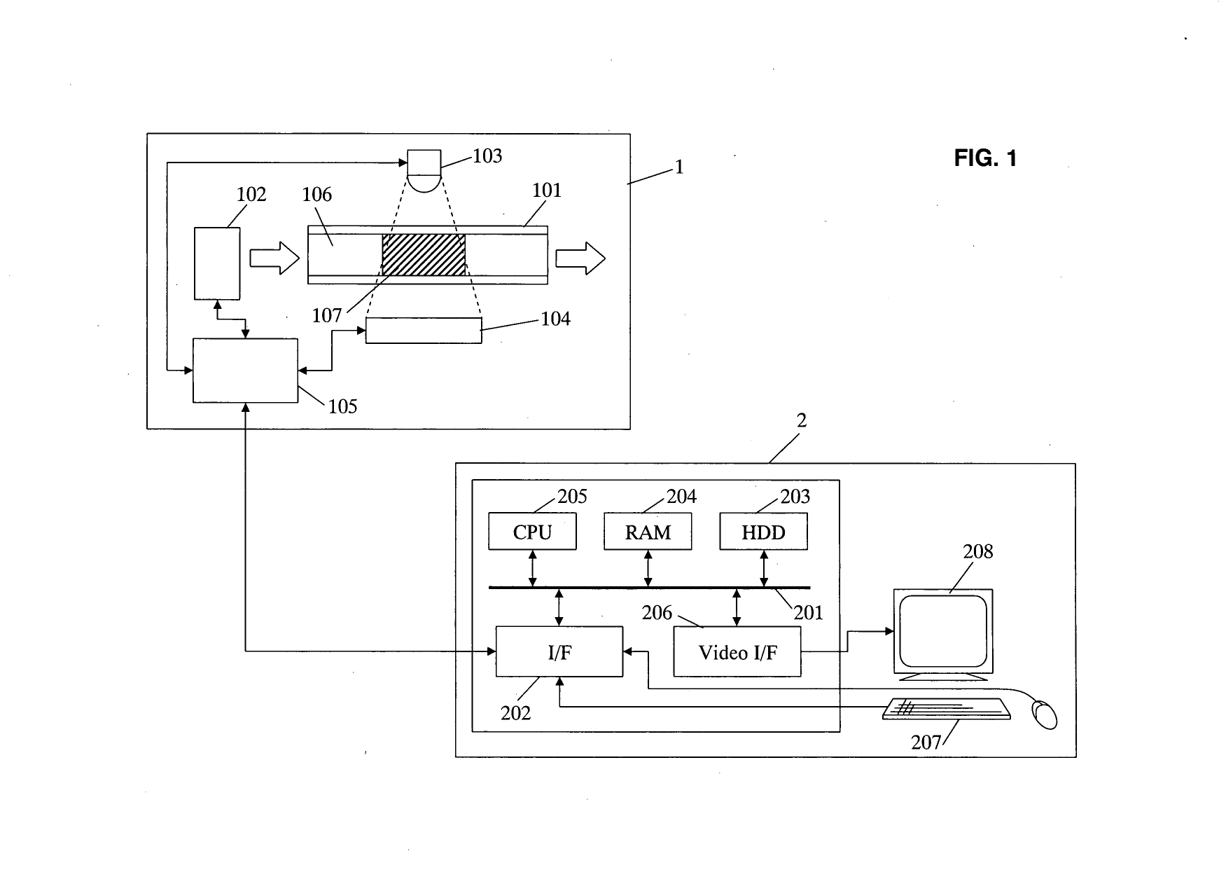

FIG. 1 is a diagram for explaining an outline configuration of screening measuring system in the embodiment of the present invention. This screening measuring system is equipped with a measuring part 1 and a data processor 2.

The measuring part 1 is equipped with a measurement cell 101, a pump 102, an excitation light source 103, a light power detector, and a controller 105. The measurement cell 101 has a flow channel 106 where a sample solution flows. This flow channel 106 is filled up with a carrier 107 where antigen derivatives for acquiring antibodies of dioxins, which are antigens, are solid-phased.

For the pump 102, a constant rate pump, such as a syringe pump, can be used. A sample solution, where a solution containing antibodies is mixed, is poured into the flow channel 106 of the measurement cell 101 by the pump 102. With this design, the measuring part 1 can measure the antibodies acquired by the carrier 107.

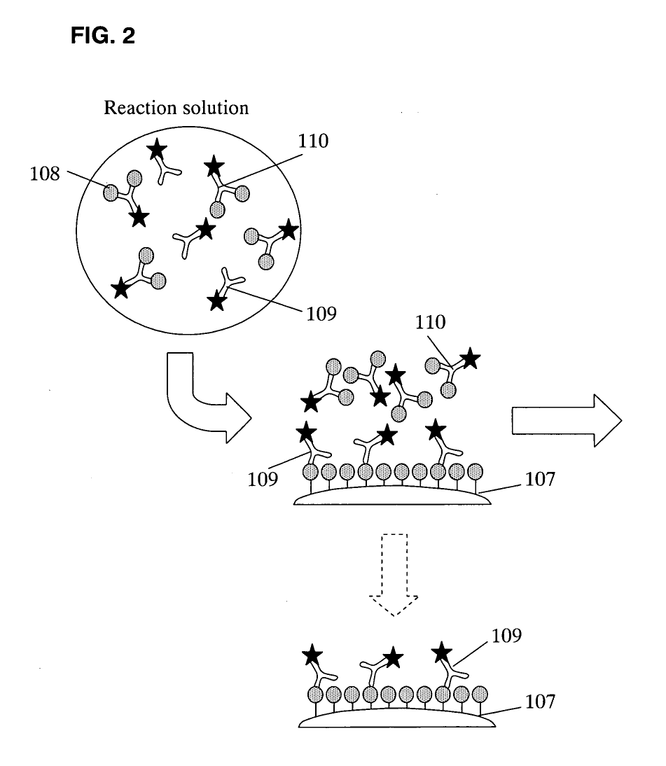

FIG. 2 is a diagram for explaining a measurement principle. An unknown amount of dioxins 108 comprising antigens, are contained in an environmental sample solution. In order to quantify the dioxins 108, the environmental sample solution and a solution containing a known amount of labeled antibodies 109 are mixed, binding the dioxins 108 in the environmental sample solution and a portion of labeled antibodies 109 due to the antigen-antibody complex reaction, thereby forming antigen-antibody complexes 110. The reaction solution contains the antigen-antibody complexes 110 and the unreacted labeled antibodies 109. The reaction solution is poured into the flow channel 106 by the pump 102. When the reaction solution is poured into the flow channel 106, the unreacted labeled antibodies 109 are acquired by the carrier 107. The antigen-antibody complexes 110 pass through the carrier 107, and are discharged from the measurement cell 101. When the total amount of the labeled antibodies 109 contained in the reaction solution is known, if the amount of the labeled antibodies 109 acquired by the carrier 107 is measured, the amount of the dioxins 108 can be obtained by subtracting the measured amount from the total amount of the labeled antibodies 109. In order to measure the amount of the antibodies 109 acquired by the carrier 107, the antibodies 109 are labeled by a fluorescent reagent.

For the antibodies 109, an antibody (see Japanese Patent Application No. 2004-003234) showing high reactivity with pentachlorodibenzofuran or hexachlorodibenzofuran, which is highly correlated with a total TEQ amount in dioxins derived from burning, such as 2,3,4,7,8-PeCDF.

Using the measurement principle and antibodies mentioned above enables very prompt measurement compared to the conventional simple measuring method. “Procedures”, which take several hours with a conventional immunoassay measurement, can be completed in several minutes.

The excitation light source 103 in FIG. 1 irradiates an excitation light to the carrier 107. The labeled antibodies 109 acquired by the carrier 107 produce fluorescence due to the excitation light.

The light power detector is arranged in a position facing the excitation light source 103 across the measurement cell 101. The fluorescence from the labeled antibodies 109 enter into this light power detector. The light power detector contains a photoelectric element, and can output electrical time series signals according to the fluorescent intensity. The light power detector samples the electric signals at appropriate time intervals, and outputs sensor data including a numerical data column indicating the fluorescent intensity to the controller 105.

The controller 105 controls the entire measuring part 1 including the pump 102, the excitation light source 103 and the light power detector. The controller 105 controls the pump 102 and the excitation light source 103, and allows the light power detector to output the sensor data.

The measuring part 1 conducts calibration and the regular measurement due to the control by this controller 105, and washing and other operations are also automatically conducted. Before the regular measurement, the measuring part 1 conducts Bo measurement and internal standard sample measurement. In the Bo measurement, the fluorescent intensity of a sample that does not contain dioxins, which are subject substances, is measured. In the internal standard sample measurement, the measurement is conducted using an internal standard sample. This measurement is a measurement for calibration. In the regular measurement, multiple environmental samples containing dioxins can be sequentially measured.

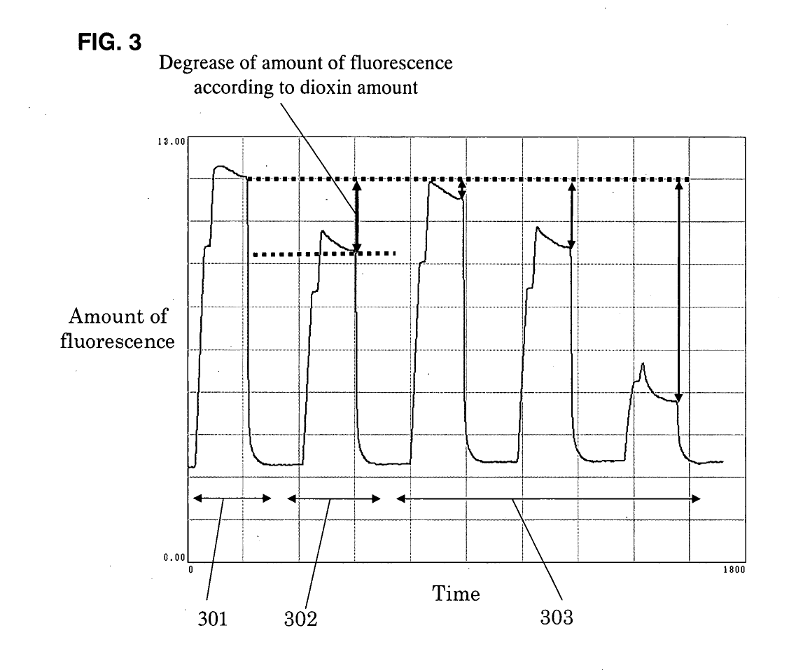

FIG. 3 shows an example of measurement data. In this graph, the horizontal axis indicates the time, and the vertical axis indicates the fluorescent intensity. Data during a period of time 301 corresponds to the Bo measurement; data during a period of time 302 corresponds to the internal standard sample measurement; and data during a period of time 303 corresponds to the regular measurement. In this example, three environmental samples were measured. The difference of the fluorescent intensity between the Bo measurement and the regular measurement is derived from the concentration of dioxins. In the regular measurement, since a portion of the antibodies 109 form the antigen-antibody complexes 110, the unreacted antibodies 109 become lessened. Consequently, the value of fluorescent intensity becomes smaller in the regular measurement compared to the BO measurement.

When obtaining the sensor data from the light power detector due to the execution of this measurement sequence, the controller 105 outputs the sensor data to the data processor 2.

The data processor 2 in FIG. 1 determines whether or not the environmental samples require any other measurement(s) based upon the data indicating the pre-determined range including the reference value with regard to dioxins, which are subject substances, and time-series sensor signals from the light power detector. Whether or not the measurement by the GC/MS method is required is determined herein. Quantification can be conducted not only by the GM/MS method but also by other measuring methods. When the subject substances is a dioxin, for example, an exhaust reference value of a new small-sized furnace (burning capacity: 2 t/h) to exhaust gas, 5 ng-TEQ/m³N, can be used as a reference value. For the reference value, various values, such as an environmental reference value or a search index value, can be used in addition to the exhaust reference value. The pre-determined range including the reference value is, for example, a range corresponding to 0.5 times of value to double value of the reference value. If the reference value is 5 ng-TEQ/m³N, the range from 2.5 ng-TEQ/m³N to 10 ng-TEQ/m³N can be provided for the toxicity equivalent quantity.

For the data processor 2, exclusive hardware can be used, and a general-purpose computer can be used. A general-purpose computer is used at this time. In the example of FIG. 2, a bus 201 of the computer connects an interface 202, a HDD 203, a RAM 204, a CPU 205 and a video interface 206.

The interface 202 connects the controller 105 to the data processor 2. The data column of the sensor data obtained by the light power detector is sequentially entered to the data processor 2 by this interface 202. Further, in this example, an input device 207, such as a keyboard or a cursor device, is also connected to the interface 202, by which a user can provide instructions to the data processor 2 using this input device 207.

The HDD 203 can store a determination program for evaluating the necessity to confirm whether or not to satisfy the reference value using the GC/MS method. Further, the data indicating the pre-determined range including an environmental value is also pre-stored in this HDD 203.

The RAM 204 can be utilized for temporarily storing a program or data read from the HDD 203.

When receiving, for example, a control signal from the controller 105 or an instruction from a user, the CPU 205 reads out the determination program from the HDD 203 and operates the computer according to the instructions of the determination program. With this design, the data processor 2 realizes a function for the determination and a function to output the determined result. The CPU 205 reads out data indicating the pre-determined range from the HDD 203 for realizing the determination function, and temporarily stores the read data on the RAM 204. The data column of the sensor data is also temporarily stored in the RAM 204.

For the determination, the CPU 205 predicts a highest value of the sampling value in the regular measurement from the data of the start time of the regular measurement for each sample, and determines whether or not the predicted value is within the pre-determined range. In order to obtain the predicted value, the CPU 205 specifies the data of the start time of the regular measurement for each sample from the sensor data on the RAM 204. If the variation of the sampling values is calculated after moving along the time-series direction, the data of the start time of the regular measurement can be identified. As in the example in FIG. 3, there are some periods of time when the fluorescent intensity scarcely changes during each measurement, and the variation remains in the vicinity of zero during the periods of time. If a threshold value greater than zero, is set, the data of start time can be identified according to whether or not the sampling value exceeds the threshold value. The variation may be calculated using a sampling value and one or more prior sampling values, for example, every time said sampling value is newly entered.

Instead of calculating the variation, the controller 105 may include header data indicating the start time in the sensor data. For example, if data about a start time of measurement sequence, a start time of the regular measurement and a sampling time is included in the header data; a sampling value (when the value was entered is known) can be identified as the data for the start time of the regular measurement.

If the data for the start time of the regular measurement is identified as described above, the CPU 205, for example, calculates the variation using the data of the start time and the data immediately thereafter or multiple sampling values thereafter. When the variation is calculated, for example, the preset time interval is multiplied by the variation, and if the multiplied value is added to the sampling value of the start time, the highest value of the sampling values in the regular measurement is predicted.

When calculating the predicted value of the highest value, the CPU 205 determines whether or not the predicted value is within the pre-determined range. The value is compared as whether or not the predicted value is less than an upper limit value and greater than a lower limit value of the pre-determined range herein. When the predicted value is obtained as the fluorescent intensity, a converted value as the fluorescent intensity is used for the pre-determined range, as well. If the predicted value is obtained as a toxicity equivalent quantity, the value within the pre-determined range relative to the toxicity equivalent quantity is used.

When having determined that the predicted value is within the pre-determined range, the CPU 205 determines that it is necessary to confirm whether or not the sample satisfies the reference value using the GC/MS method, and if the predicted value is not within the pre-determined range, it determines that no further confirmation is necessary.

As described above, instead of determining according to whether or not the predicted value is within the pre-determined range, the CPU 205 may determine whether or not an actual sampling value is within the pre-determined range.

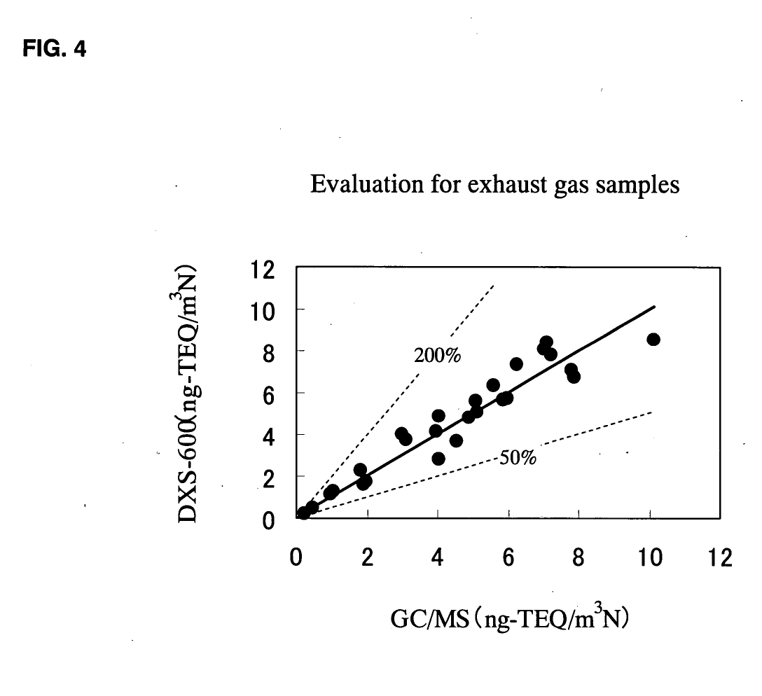

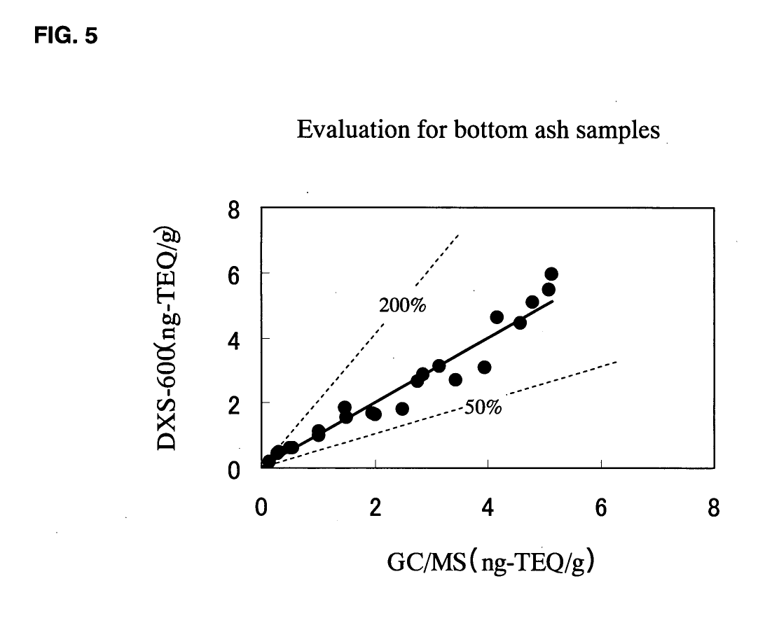

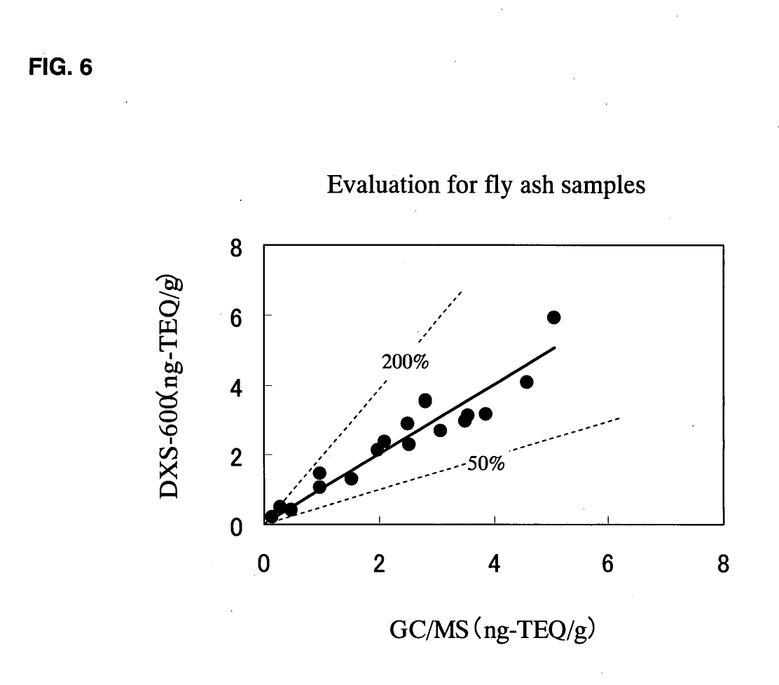

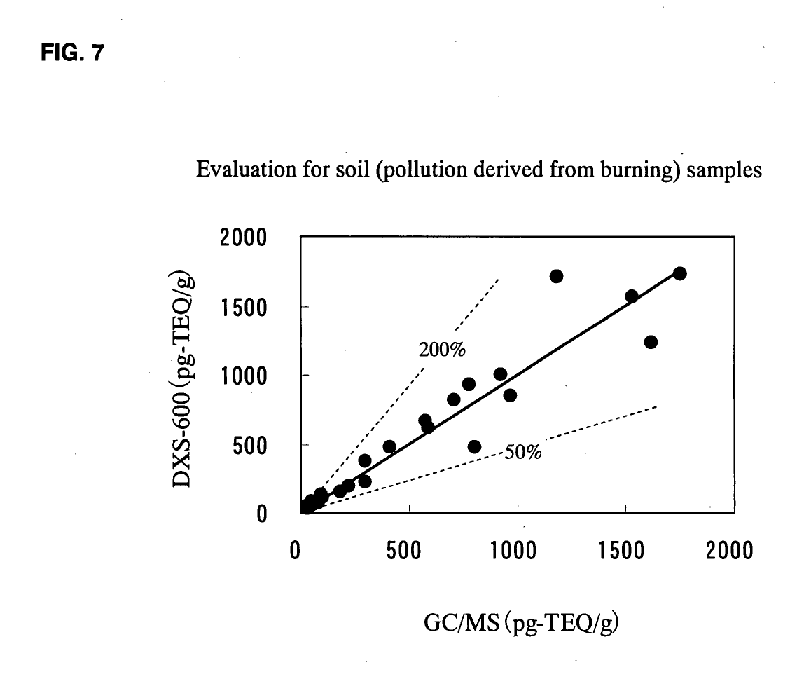

FIG. 4 to FIG. 7 show a correlation between the measuring method of the embodiment and the GC/MS method in the case of determining that it is necessary to confirm using the GC/MS method, respectively. FIG. 4 shows results of evaluation for exhaust gas samples; FIG. 5 shows results of evaluation for bottom ash samples; FIG. 6 shows results of evaluation for fly ash samples; and FIG. 7 shows results of evaluation for contaminated soil samples derived from burning. The black dots in each graph indicate samples, respectively. Although correlation values close to 1 are obtained with all types of samples, a divergence may be generated to analysis values between the measuring method of the embodiment and the GC/MS method depending upon a relative proportion of isomers. Consequently, even when the measured result is slightly smaller than a reference value with the measuring method of the embodiment, as long as the measurement result is within the pre-determined range including the reference value, it is possible that the measurement result exceeds the reference value with the GC/MS method. If it is determined using a threshold value, which is set by corresponding to the reference value, without using the pre-determined range, the determination may become false. Identification of a sample, which may exceed the reference value using the pre-determined range, enables the restraint of risk. It is sufficient that the pre-determined range can identify the sample(s), which may exceed the reference value, and it shall not limit the range from 0.5 times to 2 times.

The video interface 206 in FIG. 1 displays a determined result on the display 208 according to the instruction of the CPU 205. The measurement result of the measuring part 1 is also displayed herein. The CPU 205 creates image signals for the monitor screen from the determined result and the measurement result, and supplies the image signals to the video interface 206. The video interface 206 displays the monitor screen on the display 208 according to the image signals.

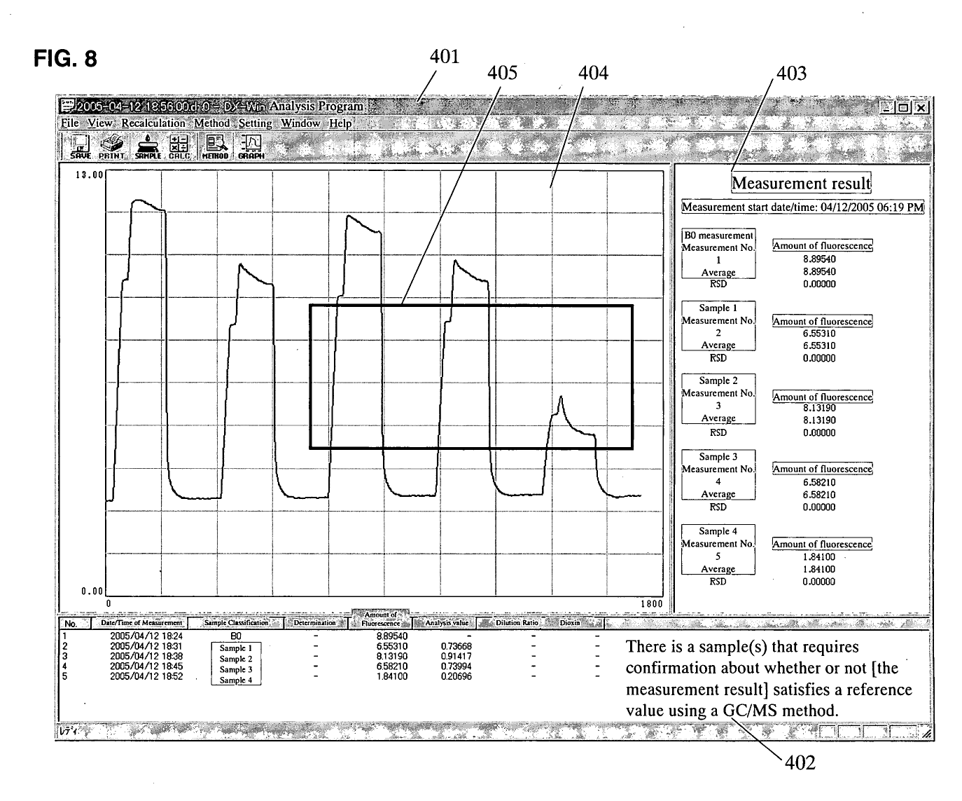

FIG. 8 shows an example of the monitor screen. A monitor screen 401 has a determination display part 402, a measurement result display part 403 and a sensorgram display part 404. The determination display part 402 displays the determined result. In this example, a message, “there is a sample(s) that requires a confirmation about whether or not the measurement result satisfies the reference value using the GC/MS method” is displayed, by corresponding to the determination where it is necessary to confirm whether or not the reference value is satisfied.

Since the measurement is promptly conducted as described above, by referring to this display, a user can instantly recognize the necessity of measurement using the GC/MS method.

The measurement result display program 403 displays data of the measurement result with numbers. The sensorgram display part 404 displays a sensorgram obtained by the measurement. A rectangular FIG. 405 on the sensorgram display part 404 corresponds to the pre-determined range. The CPU 205 creates an image of this rectangular 405 using the data on the RAM 204. The user can visually recognize the necessity of another measurement using the GC/MS method instantly, even by the display of the pre-determined range including the reference value of the subject substance and the time-series signals of the measurement result to be comparable. The user can identify the sample(s), where quantitative determination of total dioxin amount and a contaminant source should be identified, in the early stage. As a result, reduction of time for the entire analysis and reduction of the cost can be accomplished.

Further, if the measured value is not within the pre-determined range, whether or not the sample meets the criteria can be identified without depending upon the confirmation using the GC/MS method. If the concentration of dioxins is a lower limit value of the pre-determined range or less (if the fluorescent intensity is an upper limit value of the pre-determined range or greater), it meets the criteria. In the meantime, if the concentration of dioxins is an upper limit value of the pre-determined range or greater (if the fluorescent intensity is a lower limit value of the pre-determined range or less), it does not meet the criteria. The user can also determine this easily and in the early stage from the display of the display 208, which may be designed such that if the concentration of the dioxins is the lower limit value of the pre-determined range or less, the CPU 205 creates a message, “The sample is lower than the pre-determined range (50% of the reference value or less),” and if the concentration is the upper limit value of the pre-determined range or greater, it creates another message, “The sample is higher than the pre-determined range (200% of the reference value or greater). Careful examination is recommended,” and the message is displayed on the display 208.

As described above, whether or not the measurement result of the sample is within the pre-determined range is predicted based upon the time-series signals in the early of sample measurement, if the measured value is not within the pre-determined range, the measurement of the sample can be stopped, as well. The measurement is stopped, for example, when the concentration of the dioxins is an upper limit value of the pre-determined range or greater. If it is determined that the measured value is not within the pre-determined range according to the prediction based upon the time-series signals in the early of measurement, the CPU 205 transmits a control signal to the controller 105 by following the instruction of the program. When receiving the control signal, the controller 105 stops the sample measurement operation thereafter, and moves on to the measurement of another sample. Specifically, the repetitive measurement of the sample is canceled, and after washing, the measurement of the next sample is started.

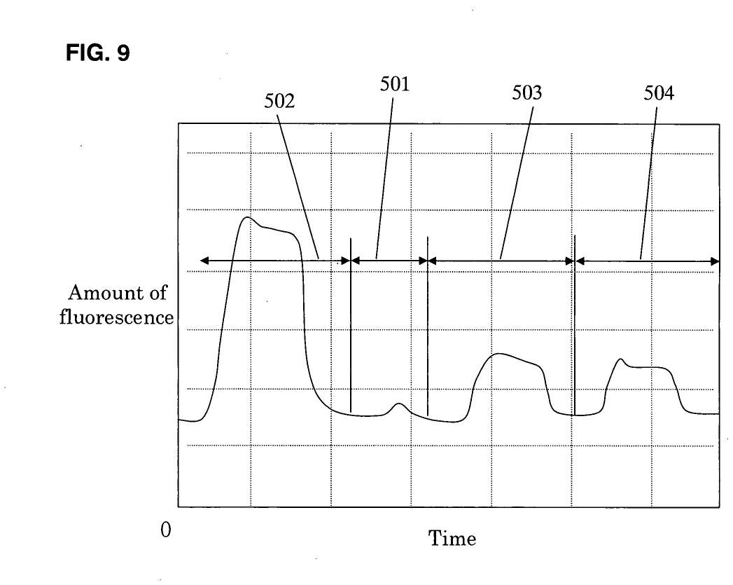

FIG. 9 shows an example of a screen displaying a measurement result when the sample measurement is stopped halfway. In this example, the measurement is stopped in a period of time 501, which is the second from the left. If the measurement is stopped as mentioned above and the measurement is moved on to a next sample, the length of the period of time 501 becomes shorter than other periods of time 502, 503 and 504 for other samples. In other words, stopping the measurement enables reduction of the time required for the measurement, which is especially effective in the case of measuring many samples.

The embodiment does not limit the technical scope of the present invention, and the present invention is variously modifiable and applicable within its scope other than the described ones. For example, hardware of the data processor 2 is composed with exclusive hardware, and incorporation of the hardware into the measuring part 1 may realize the function mentioned above by the integrated hardware.

In the embodiment described above, the fluorescent intensity is measured. However, absorbancy can be measured according to a label, and other physical quantity can be measured, as well.

Further, in the embodiment described above, both the determination of the necessity of measurement by the GC/MS method or another method and the display of the measurement result and the pre-determined range to be comparable are conducted. However, either one may be conducted, and the user can make a determination visually with either.

Further, another antibody can be used for the antibody for detecting dioxins. In addition, the present invention is applicable not only to the environmental samples containing dioxins, but also to screening measurement for other environmental samples that require the infinitesimal quantity analysis.

### INDUSTRIAL APPLICABILITY

The device and the method for screening measurement of environmental samples of the present invention have a superior effect where it becomes possible to promptly determine the selection of environmental samples, and are useful for screening measurement of environmental samples containing dioxins, other environmental pollutants or persistent organic pollutants.

1. A screening measuring method for an environmental sample, comprising the steps of:
measuring the environmental sample;
determining whether or not a measurement result is within a pre-determined range including a reference value based upon a time-series signal of the measurement result; and
outputting the measurement result.

2. A screening measuring method for an environmental sample, comprising the steps of:
pouring a sample solution in which a known amount of antibody of a subject substance being an antigen is mixed, into a measurement cell having a flow channel for the sample solution, wherein an antigen derivative for acquiring the antibody is arranged in the flow channel;
measuring the antibody acquired by the antigen derivative;
determining whether or not a measurement result is within a given pre-determined range including a reference value based upon a time-series signal of the measurement result; and
outputting the measurement result.

3. The screening measuring method according to claim 2, wherein the step for determination comprises the step of predicting whether or not the measurement result of the sample is within a pre-determined range based upon the time-series signal in the early of sample measurement, and the determination is conducted based upon the predicted result.

4. The screening measuring method according to claim 3, further comprising the step of moving on to the measurement of another sample, in case. of predicting that the measurement result of the sample is not within the pre-determined range.

5. The screening measuring method according to claim 1, wherein in the step for output, in case of determining that the measurement result is within the pre-determined range in the step for determination, necessity to confirm whether or not to satisfy a reference value is displayed.

6. A screening measuring method for an environmental sample, comprising the steps of:
pouring a sample solution in which a known amount of antibody of a subject substance being an antigen is mixed, into a measurement cell having a flow channel for the sample solution, wherein an antigen derivative for acquiring the antibody is arranged in the flow channel;
measuring the antibody acquired by the antigen derivative; and
displaying a pre-determined range including a reference value of the subject substance and a time-series signal of the measurement result to be comparable.

7. A screening measuring device, comprising:
a measurement cell that has a flow channel for a sample solution, wherein an antigen derivative for acquiring an antibody of a subject substance being an antigen is arranged in the flow channel;
a unit configured to measure the antibody acquired by the antigen derivative by pouring the sample solution in which a known amount of the antibody is mixed, into the measurement cell;
a unit configured to store data indicating a pre-determined range including a reference value for the subject substance;
a unit configured to determine whether or not the measurement result is within the pre-determined range based upon a time-series signal of the measurement result; and
a unit configured to output the measurement result.

8. The screening measuring device according to claim 7, wherein the unit for determination predicts whether or not the measurement result of the sample is within the pre-determined range based upon the time-series signal in the early of sample measurement, and conduct the determination based upon the predicted result.

9. A screening measuring device, comprising:
a measurement cell having a flow channel for a sample solution, wherein an antigen derivative for acquiring an antibody of a subject substance being an antigen is arranged in the flow channel;
a unit configured to measure the antibody acquired by the antigen derivative by pouring the sample solution in which a known amount of the antibody is mixed, into the measurement cell;
a unit configured to store data indicating a pre-determined range including a reference value for the subject substance; and
a unit configured to display the pre-determined range including a reference value for the subject substance and a time-series signal of the measurement result to be comparable using the stored data of pre-determined range and the time-series signal of the measurement result.
